(19) United States
(12) Patent Application Publication
(10) Pub. No.: US 2013/0310689 A1
(43) Pub. Date: Nov. 21, 2013

(54) ULTRASOUND DIAGNOSTIC DEVICE

(71) Applicant: Morio Nishigaki, Kanagawa (JP)

(72) Inventor: Morio Nishigaki, Kanagawa (JP)

(73) Assignee: PANASONIC CORPORATION, Kadoma-shi, Osaka (JP)

(21) Appl. No.: 13/981,470

(22) PCT Filed: Aug. 27, 2012
(86) PCT No.: PCT/JP2012/005350

§ 371 Date: Jul. 24, 2013

(30) Foreign Application Priority Data
Aug. 31, 2011 (JP) 2011-189277

(51) Int. Cl. A61B 8/00

(52) U.S. Cl. CPC A61B 8/4483
USPC 600/437

(57) ABSTRACT

An ultrasound diagnostic device includes: a transmission pulser that applies a driving pulse output signal to an ultrasonic transducer so as to cause the ultrasonic transducer to irradiate ultrasound; and a trigger signal generator that generates a trigger signal that controls the transmission pulser to output the driving pulse output signal. The trigger signal generator adds, to the trigger signal in a predetermined term after completion of a driving term of the ultrasonic transducer, a plurality of trigger pulses that control an amplitude of an ultrasonic transducer output signal generated upon completion of the driving term so as to attenuate gradually. With this configuration, a free vibration of the ultrasonic transducer in a transient response can be suppressed with the minimum number of circuit materials.

### TECHNICAL FIELD

The present invention relates to an ultrasound diagnostic device used for medical diagnoses, and in particular relates to a technology to generate ultrasonic pulses for irradiation of a body in an ultrasound diagnostic device.

### BACKGROUND ART

In the medical field, ultrasound diagnostic devices are used for performing image diagnoses on target areas. FIG. 14 is a block diagram showing a schematic configuration of an ultrasound diagnostic device. An ultrasonic transducer 1 performs conversion between ultrasound and electricity, and is connected via a cable 2 to a transmission pulser 3 and a reception circuit 5 that are provided in a main body of the ultrasound diagnostic device. The transmission pulser 3 supplies to the ultrasonic transducer 1, via the cable 2, driving pulse output signals that are driving pulse trains of high-voltage electrical pulses. The generation timing of driving pulse output signals is determined based on trigger signals generated by a trigger signal generator 104. The reception circuit 5 amplifies echo signals obtained by converting ultrasound reflected inside a body into electricity by means of the ultrasonic transducer 1, focuses beams, and the like. A signal processor 6 processes output signals of the reception circuit 5, and calculates amplitude information, flow information, etc. A display unit 7 displays images and information processed by the signal processor 6.

Further, although not illustrated, an operation unit for allowing an operator to operate the ultrasound diagnostic device and a control unit for performing the overall control are contained in the ultrasound diagnostic device.

In the configuration of the ultrasound diagnostic device, there are several kinds of waveforms of driving pulse output signals to be generated from the transmission pulser 3. Selecting the waveform to be output determines the internal configuration of the transmission pulser 3.

The transmission pulser 3 can be regarded as a sort of amplifier that converts trigger signals having been output from the trigger signal generator 104 into driving pulse output signals of high-voltage electrical pulses. As amplifiers for applications other than ultrasound, a linear amplifier is used generally. Since the linear amplifier consumes much electric power inside a circuit and causes problems such as heat generation, it rarely is used in ultrasound diagnostic devices. An amplifier generally used as the transmission pulser 3 of the ultrasound diagnostic device is a circuit with FET switches. FIG. 2 is a schematic circuit diagram of the transmission pulser 3 with FET switches. FIG. 7 is a timing chart showing trigger signals and a driving pulse output signal in the circuit of FIG. 2.

The waveforms of driving pulse output signals in the switch-type circuit with FET switches are classified roughly into a unipolar waveform whose amplitude is on either a positive side or a negative side, and a bipolar waveform whose amplitude is on both the positive side and the negative side. The configuration shown in FIG. 2 is a type generating a bipolar waveform. By turning ON a first FET switch 11, a line to the ultrasonic transducer 1 is connected to a positive voltage source. By turning ON a second FET switch 12, the line to the ultrasonic transducer 1 is connected to a negative voltage source. As shown in FIG. 7, by turning ON the first FET switch 11 and the second FET switch 12 alternately, the transmission pulser 3 generates driving pulses.

Meanwhile, regarding the unipolar waveform, by alternately turning ON two FET switches for connection of an output to the positive voltage source (or negative voltage source) or a ground, driving pulse output signals are generated. In the circuit of FIG. 2, the waveform of the unipolar type can be generated by connecting the negative voltage source to the ground and turning ON the two FET switches alternately.

In the transmission pulser that generates a unipolar waveform, since signals obtained by inverting ON/OFF of first trigger signals can be used as second trigger signals, only one kind of trigger signal is required practically, whereby the circuit scale can be downsized. However, due to the narrow choice of the frequency of driving pulse output signals to be generated, recently, such a generator is less likely to be used.

Meanwhile, since the transmission pulser that generates a bipolar waveform is composed of two FET switches for connection to the positive voltage source and the negative voltage source, both the FET switches are turned OFF so as not to apply a voltage to an ultrasonic probe after completion of generation of driving pulse output signals. In this case, the output of the transmission pulser becomes high impedance, which causes a problem of generating a transient waveform.

FIG. 8A is a diagram showing a driving pulse output signal in a state where the output of the transmission pulser 3 is open. After completion of a driving term, a charge leaks little by little in a transient phenomenon term, whereby a voltage gradually approaches a ground level. FIG. 8B is a diagram showing a driving pulse output signal in a state where the ultrasonic transducer 1 is connected to the transmission pulser 3. Since the ultrasonic transducer 1 serves as a sort of a resonance circuit, an amplitude of the driving pulse output signal attenuates gradually while vibrating after the driving term.

In order to suppress such a transient phenomenon, a configuration shown in FIG. 9A is proposed (for example, see JP 11(1999)-342127 A). In this configuration, a first FET switch 101 and a second FET switch 102 are connected in series between a positive voltage source and a negative voltage source, and a node between the first FET switch 101 and the second FET switch 102 is connected to the ultrasonic transducer 1. A third FET switch 103 with one end grounded is connected to the node.

FIG. 9B is a timing chart showing trigger signals to be input to the respective FET switches and a driving pulse output signal to be supplied to the ultrasonic transducer 1. As can be seen from the driving pulse output signal, this arrangement can control the transient phenomenon to some extent. However, this configuration increases the number of FET switches and the number of control signals for the increased FET switches.

Further, there has been known a method of releasing a charge accumulated after the driving term by generating extremely short trigger signals from the trigger signal generator after the driving term (for example, see JP 2002-315748 A). However, as can be seen from a timing chart of this method shown in FIG. 10, a transient response term is shortened but remains. The transient response term remains significant especially when the ultrasonic transducer 1 is connected to the transmission pulser 3.

Next, imaging using harmonics generated through nonlinear propagation in a body will be described. As compared with common methods, imaging using harmonics can improve the resolution in a lateral direction. Therefore, in areas where reflection is little such as a blood vessel and a heart chamber, images less affected by nearby tissues can be observed.

There are several methods of performing harmonic imaging. For example, a method has been known in which reception signals are high-pass filtered so that fundamental wave components are removed and only harmonic components are extracted.

In the ultrasonic transducer 1 used for the ultrasound transmission/reception, there is a part (band) where the transmission/reception efficiency is favorable. The range is not so wide, which is about 70% (relative bandwidth) with respect to a center frequency of the ultrasonic transducer 1. Generally, the ultrasound transmission/reception is performed substantially at the center of the band. However, in imaging using harmonics, a lower side of the band of the ultrasonic transducer 1 is used for transmission and a higher side of the band of the ultrasonic transducer 1 is used for reception. Further, since the harmonic imaging is performed using harmonics generated inside a body, the image quality deteriorates if the driving pulse output signals themselves contain harmonics.

Ultrasound diagnostic devices include modes as a Doppler blood flow meter and a color-flow blood flow video device. The detailed description of the Doppler blood flow meter and the color-flow blood flow video device will be omitted because their principles and configurations have already been known widely. These devices transmit and receive ultrasound in the same direction plural times, extract a phase change portion in reception signals, and display it as a blood flow. In the Doppler blood flow meter and the color-flow blood flow video device, an amount of phase change is converted into a flow velocity. Even if the flow velocity is the same, the amount of phase change varies when the frequency used for transmission/reception differs. Specifically, the higher the frequency is, the more the amount of phase change increases.

Further, when the ultrasound transmission/reception is performed using a comparatively high frequency, e.g., about 10 MHz, scattering in body tissues sometimes becomes large depending on a subject and an area to be tested, and only images with much acoustic noise can be obtained. In this case, it is possible to reduce such acoustic noise by lowering the frequency of driving pulse output signals.

Conversely, in some cases, for the purpose of improving an azimuth resolution, the frequency of driving pulses is increased using the ultrasonic transducer 1 having a relatively low frequency band so that the frequency of ultrasonic pulses generated from the ultrasonic transducer 1 becomes higher than the center frequency of the ultrasonic transducer 1. In other words, the center frequency of the ultrasonic transducer 1 is not always used.

As described above, in the case of the transmission pulser 3 shown in FIG. 2, when both the FET switches are turned OFF after the driving term, the transient response (transient response term) continues as mentioned above. As shown in FIGS. 11A to 11C, the transient response exhibits a period TO of a free vibration of the ultrasonic transducer 1, regardless of the frequency of the driving term (period: TL>T0, TH<T0). That is, the frequency becomes one near a center frequency f0 of the ultrasonic transducer 1.

FIG. 12A shows a frequency spectrum in a case where the frequency of the driving term is the period TO (frequency: f0) of the free vibration of the ultrasonic transducer 1 as shown in FIG. 11A. The spectrum has a single-peak characteristic.

Meanwhile, FIG. 12B shows a frequency spectrum in a case where the ultrasonic transducer 1 is driven at a frequency fL lower than the period T0 (frequency f0) of the free vibration of the ultrasonic transducer 1 as shown in FIG. 11B. The spectrum contains high frequency components other than components near the driving frequency fL.

In contrast, FIG. 12C shows a case where the ultrasonic transducer 1 is driven at a frequency (fH) higher than the period T0 (frequency f0) of the free vibration of the ultrasonic transducer 1 as shown in FIG. 11C. The spectrum contains low frequency components other than components near the driving frequency fH.

Especially, in the harmonic imaging, the image quality deteriorates if driving pulse output signals themselves contain high frequencies. Also in the Doppler blood flow meter and the color-flow blood flow video device, if the driving pulse output signals contain high frequencies, the reception signals also contain high frequencies. This prevents accurate calculation of a blood flow velocity.

Moreover, in the case where much acoustic noise is generated depending on a subject and an area to be tested after performing transmission using a relatively high frequency, even if an operator tries to use a transmission frequency lower than the center frequency of the ultrasonic transducer, high frequencies are contained due to the above-discussed transient phenomenon of the ultrasonic transducer 1. Hence, desired effects cannot be obtained.

Conventional techniques for solving these problems will be discussed below. In the case of the transmission pulser 3 shown in FIG. 9A, effects can be recognized as compared with the configuration with two FET switches. However, sufficient effects cannot be expected because an impedance of the FET switch 103 to be connected to a cable and a ground cannot be matched with an impedance of the ultrasonic transducer. Further, also in the mode shown in FIG. 10, sufficient effects cannot be expected.

In order to solve this problem, a technique using an inductor has been known (for example, see JP 8(1996)-182680 A). FIG. 13 is a block diagram showing a configuration of an ultrasonic probe using this method. By interposing a circuit including an inductor 113 and a switch 112 in parallel between a transmission pulser 114, a reception circuit 115 and an ultrasonic transducer 111, a characteristic frequency is changed. At the time of transmission, the inductor 113 is used so as to lower the characteristic frequency. At the time of reception, the switch 112 is turned ON so as to deactivate the inductor 113.

### PRIOR ART DOCUMENTS

### Patent Documents

Patent Document 1: JP 11(1999)-342127 A

Patent Document 2: JP 2002-315748 A

Patent Document 3: JP 8(1996)-182680 A

### DISCLOSURE OF INVENTION

### Problem to be Solved by the Invention

However, when adopting the configuration shown in FIG. 13, the number of circuit materials such as a switch and an inductor increases, and further switching noise is generated because switching is required at the timings of transmission and reception.

The present invention has been achieved to solve the above-described conventional problems, and the object is to provide an ultrasound diagnostic device that suppresses a free vibration of an ultrasonic transducer in a transient response with the minimum number of circuit components, thereby keeping a frequency of ultrasound after completion of a driving term at a frequency in a steady state.

### Means for Solving Problem

To solve the above problems, an ultrasound diagnostic device of the present invention includes a transmission pulser that applies a driving pulse output signal to an ultrasonic transducer so as to cause the ultrasonic transducer to irradiate ultrasound; and a trigger signal generator that generates a trigger signal that controls the transmission pulser to output the driving pulse output signal. The trigger signal generator adds, to the trigger signal in a predetermined term after completion of a driving term of the ultrasonic transducer, a plurality of trigger pulses that control an amplitude of an ultrasonic transducer output signal generated upon completion of the driving term so as to attenuate gradually.

Further, it is possible to have a configuration in which the trigger pulses cause, in the predetermined term after completion of the driving term of the ultrasonic transducer, the ultrasonic transducer output signal generated upon completion of the driving term to keep a driving period in the driving term while attenuating the amplitude gradually.

Further, it is possible to have a configuration in which the trigger pulses cause, in the predetermined term after completion of the driving term of the ultrasonic transducer, the ultrasonic transducer output signal generated upon completion of the driving term to attenuate the amplitude gradually by means of setting at least one of a pulse starting point, a pulse width, the number of pulses and a pulse pause term.

Further, it is possible to have a configuration in which the driving pulse output signal includes a positive driving pulse and a negative driving pulse, and the transmission pulser has a first switch that generates the positive driving pulse to the ultrasonic transducer and a second switch that generates the negative driving pulse to the ultrasonic transducer.

Further, it is possible to have a configuration in which the trigger signal includes a first trigger signal that controls the first switch and a second trigger signal that controls the second switch.

Further, it is possible to have a configuration in which, after completion of the driving term of the ultrasonic transducer, the trigger signal brings the first switch and the second switch into an ON state alternately at 1/2 period of the driving period and shortens time of the ON state gradually per 1/2 period of the driving period.

Further, it is possible to have a configuration in which when ON/OFF of the first switch and the second switch changes in the driving term, the trigger signal brings the first switch and the second switch into an OFF state simultaneously.

Further, the ultrasound diagnostic device can be configured to further include a switching element for replacing, with each other, the first trigger signal and the second trigger signal to be input to the first switch and the second switch, wherein pulse inversion harmonic imaging is performed by controlling the switching element.

Further, it is possible to have a configuration in which an ON/OFF frequency of the first trigger signal and an ON/OFF frequency of the second trigger signal are lower than a center frequency of the ultrasonic transducer. Moreover, it is possible to have a configuration in which an ON/OFF frequency of the first trigger signal and an ON/OFF frequency of the second trigger signal are higher than a center frequency of the ultrasonic transducer.

### Effect of the Invention

According to the present invention, by applying pulses corresponding to a frequency of a driving term after completion of the driving term, a free vibration of an ultrasonic transducer in a transient response is suppressed with the minimum number of circuit materials, thereby keeping a frequency of ultrasound after completion of a driving term at a frequency in a steady state. As a result, it is possible to create ultrasonic images with favorable image quality and to perform favorable measurements.

### BRIEF DESCRIPTION OF DRAWINGS

FIG. 1 is a block diagram showing a configuration of an ultrasound diagnostic device according to Embodiment 1 of the present invention.

FIG. 2 is a circuit diagram showing a configuration of a transmission pulser of the ultrasound diagnostic device according to Embodiment 1 of the present invention.

FIG. 3 is a timing chart showing operations of the ultrasound diagnostic device according to Embodiment 1 of the present invention.

FIG. 4 is a timing chart showing operations of an ultrasound diagnostic device according to Embodiment 2 of the present invention.

FIG. 5 is a circuit diagram showing a configuration of a transmission pulser of an ultrasound diagnostic device according to Embodiment 3 of the present invention.

FIG. 6 is a timing chart showing operations of an ultrasound diagnostic device according to Embodiment 4 of the present invention.

FIG. 7 is a timing chart showing operations of a conventional ultrasound diagnostic device.

FIG. 8A is a diagram showing a driving pulse output signal in a state where an output is open in the conventional ultrasound diagnostic device.

FIG. 8B is a diagram showing a driving pulse output signal in a state where an ultrasonic transducer is connected in the conventional ultrasound diagnostic device.

FIG. 9A is a circuit diagram showing a configuration of a transmission pulser in another conventional ultrasound diagnostic device.

FIG. 9B is a timing chart showing operations of the another conventional ultrasound diagnostic device.

FIG. 10 is a timing chart showing operations of the conventional ultrasound diagnostic device.

FIG. 11A is a diagram showing a driving pulse output signal in the conventional ultrasound diagnostic device.

FIG. 11B is a diagram showing a driving pulse output signal in the conventional ultrasound diagnostic device.

FIG. 11C is a diagram showing a driving pulse output signal in the conventional ultrasound diagnostic device.

FIG. 12A is a diagram showing a spectrum of ultrasound output from the ultrasonic transducer.

FIG. 12B is a diagram showing a spectrum of ultrasound containing low frequency components output from the ultrasonic transducer.

FIG. 12C is a diagram showing a spectrum of ultrasound containing high frequency components output from the ultrasonic transducer.

FIG. 13 is a circuit diagram showing a configuration of a transmission pulser of another conventional ultrasound diagnostic device.

FIG. 14 is a schematic block diagram showing a configuration of a conventional ultrasound diagnostic device.

### DESCRIPTION OF THE INVENTION

Hereinafter, embodiments of an ultrasound diagnostic device of the present invention will be described with reference to the drawings.

### Embodiment 1

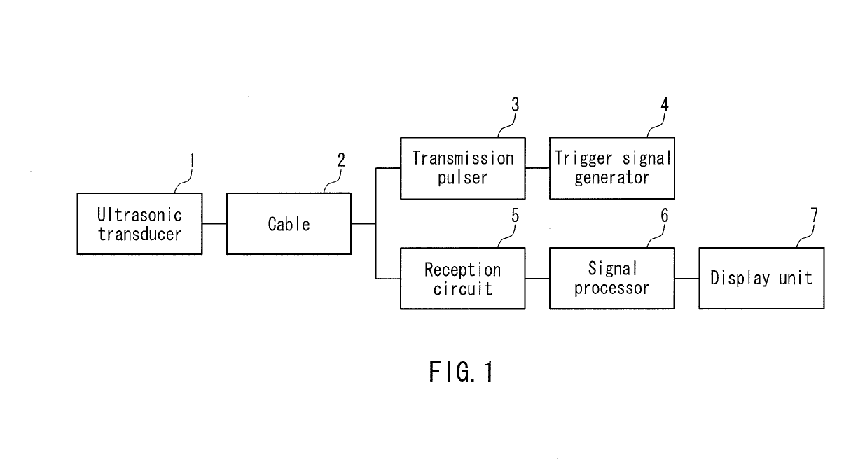

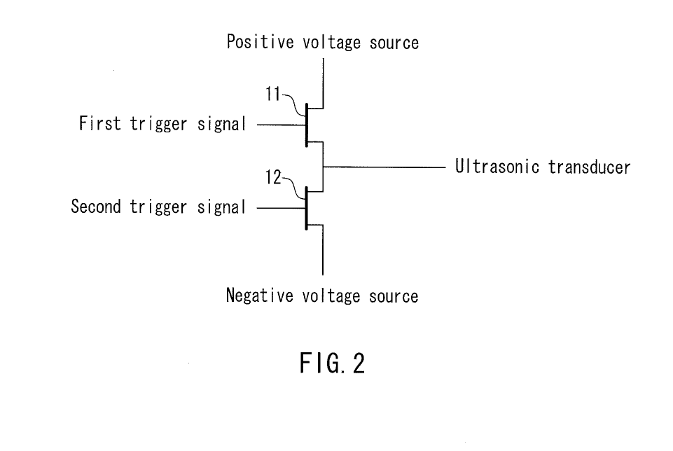

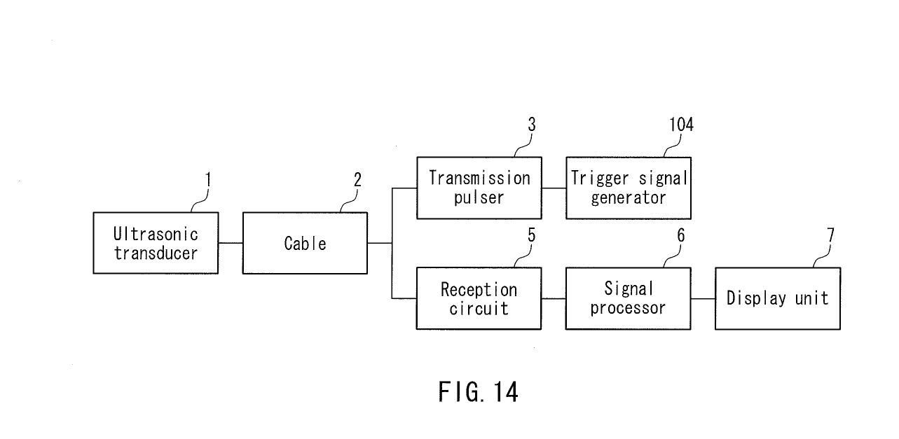

An ultrasound diagnostic device according to Embodiment 1 of the present invention has a configuration shown in FIG. 1 that is substantially identical to configurations of conventional ultrasound diagnostic devices. Constituent elements other than a trigger signal generation unit 104 are denoted with the same reference numerals as those in FIG. 14. As shown in FIG. 2, in a transmission pulser 3, a first FET switch 11 and a second FET switch 12 are connected in series between a positive voltage source and a negative voltage source. A first trigger signal is input to the first FET switch 11, and a second trigger signal is input to the second FET switch 12. A node between the first FET switch 11 and the second FET switch 12 is connected to an ultrasonic transducer 1.

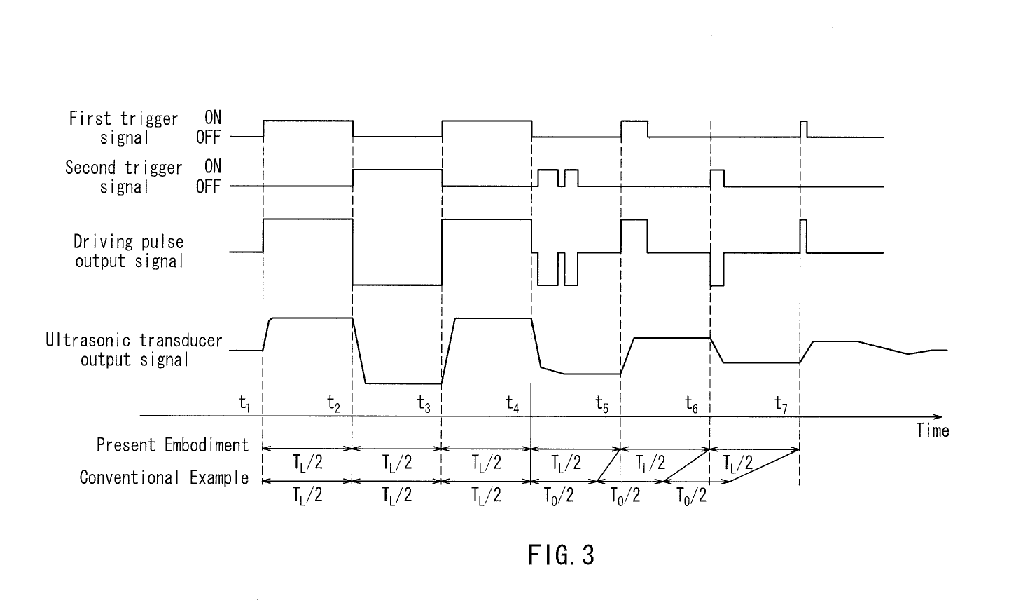

The present embodiment is characterized by the timing of trigger signals, which are generated by a trigger signal generator 4 for performing switching of the transmission pulser 3. FIG. 3 is a timing chart showing an output signal (driving pulse output signal) of the transmission pulser 3 of the ultrasound diagnostic device in the present embodiment, trigger signals, and an output signal (ultrasonic transducer output signal) from the ultrasonic transducer 1. A driving term is from times t1 to t4, and completed at time t4.

In the present embodiment, as the driving pulse output signal, a signal having a period TL longer than a period TO of the characteristic vibration of the ultrasonic transducer 1 is used. In other words, a signal having a transmission frequency lower than a frequency f0 of the characteristic vibration of the ultrasonic transducer 1 is used.

After completion of the driving term (time t4), the trigger signal generator 4 generates, as the first trigger signal and the second trigger signal, pulses shorter than TL/2 based on given data held in the trigger signal generator 4. At this time, a pulse starting point, a pulse length, the number of pulses and a pause interval between pulses are adjusted so that ultrasonic transducer output signals keep the period TL while decreasing the amplitude gradually.

Specifically, when a first trigger signal is ON and a second trigger signal is OFF in a term directly before completion of the driving term, the trigger signal generator 4 generates, in the next TL/2 term, a first trigger signal in an OFF state and a second trigger signal whose ON time is shorter than TL/2. Then, ON and OFF are repeated alternately between the first trigger signal and the second trigger signal per TL/2 term. In that case, the signals are generated so that the ON time is shortened gradually. Incidentally, as the second trigger signal between t4 and t5, a plurality of pulses may be used.

By causing the transmission pulser 3 to generate driving pulse output signals based on such trigger signals, ultrasonic transducer output signals keep the period TL while decreasing the amplitude gradually. Because of this, unnecessary high frequency components are not generated, and thus high-quality harmonic images can be obtained. In other words, in intravascular and intracardiac harmonic imaging, favorable images with less glare can be created.

Further, even when not using this technique in the harmonic imaging, signals with less acoustic noise can be obtained by the transmission/reception at low frequencies. Thereby, high-quality ultrasonic images can be obtained.

Further, in a Doppler blood flow meter and a color-flow blood flow video device, favorable measurements with few flow velocity errors can be performed.

Further, by performing the transmission/reception with a favorable azimuth resolution using a transmission frequency higher than a free vibration frequency of the transducer, it is possible to obtain high-quality images with less deterioration in a lateral direction.

Incidentally, in the present embodiment, by limiting the ON time of the FET switches, i.e., the width of the trigger pulses, ultrasonic signals for irradiation are controlled so as to have the same period as the driving term and so that the amplitude decreases gradually. However, the present embodiment is not limited to this example. By setting the pulse starting point, the pulse width, the number of pulses and the pulse pause interval, the ultrasonic signals for irradiation may be controlled to have the same period as the driving term and so that the amplitude decreases gradually.

### Embodiment 2

An ultrasound diagnostic device according to Embodiment 2 of the present invention has the same configuration as the ultrasound diagnostic device according to Embodiment 1, and is characterized by a driving timing of trigger signals that is different from the timing in Embodiment 1.

In transmission pulsers of ultrasound diagnostic devices such as the one used in Embodiment 1 of the present invention shown in FIG. 2, a large current may flow when both the FET switches are in an ON state.

A phenomenon that both the FET switches are simultaneously in the ON state occurs due to delayed timing of turning OFF the FET switch that was ON previously and early timing of turning ON the FET switch to be ON next.

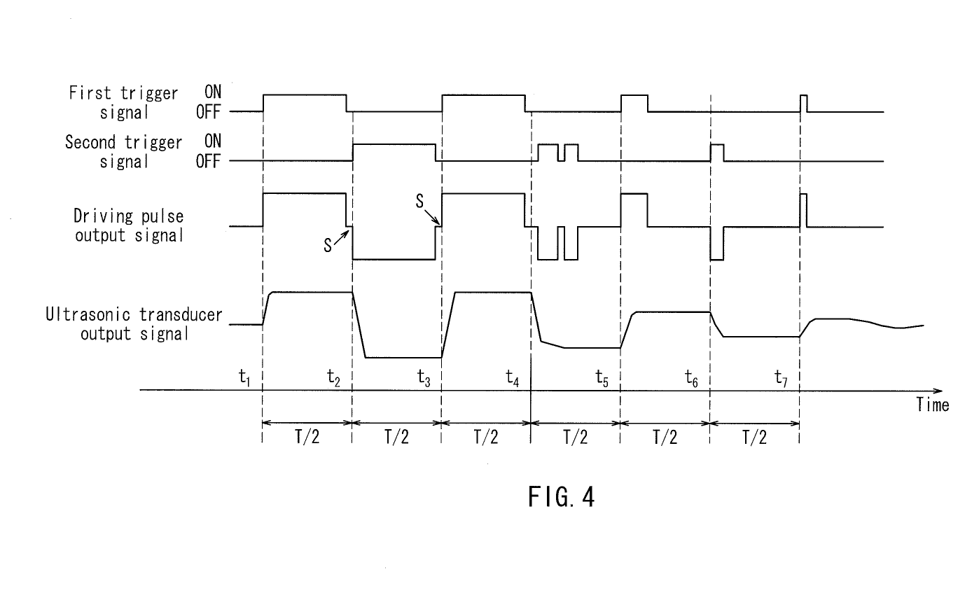

To solve this problem, trigger signals as shown in FIG. 4 are used. Specifically, a first trigger signal is turned OFF shortly before the time t2 at which a second trigger signal is turned ON. Thereby, a state in which the first trigger signal and the second trigger signal are both OFF is created. Likewise, a second trigger signal is turned OFF shortly before the time t3 at which a first trigger signal is turned ON. Thereby, a state in which the first trigger signal and the second trigger signal are both OFF is created.

In this manner, by differentiating the controlling timing of turning ON the two FET switches, it is possible to avoid the phenomenon of both the FET switches being simultaneously in the ON state.

Although the above-mentioned countermeasure may cause a moment in which both the FET switches are OFF, this rarely causes problems.

Further, as shown in FIG. 4, by using trigger signals similar to those shown in FIG. 3 after completion of the driving term, it is possible to prevent generation of frequencies other than the intended frequency during the driving term.

### Embodiment 3

An ultrasound diagnostic device according to Embodiment 3 of the present invention has a configuration in which trigger signals of the ultrasound diagnostic device according to Embodiment 1 are input via a cross point switch to the FET switches.

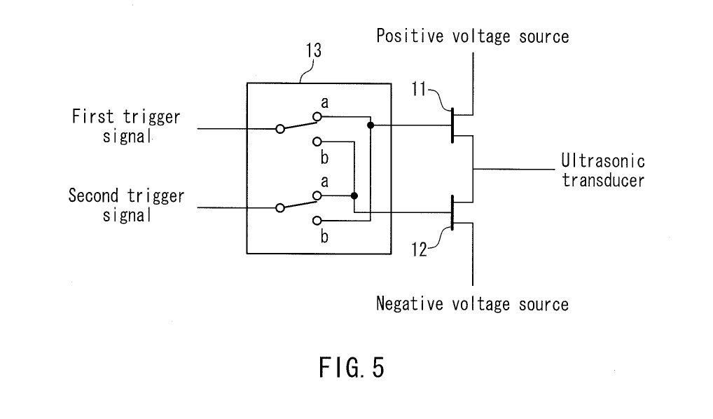

With the configurations described in Embodiments 1 and 2, ultrasonic transducer output signals can be optimized, i.e., the free vibration can be suppressed. However, in the harmonic imaging of pulse inversion mode in which ultrasounds whose phases are inverted from each other are used for irradiation, first trigger signals and second trigger signals need to be replaced with each other, which increases an amount of data for generating trigger signals for relaxation control, etc. Therefore, a large memory capacity (not shown) is required. In the present embodiment, for solving this problem, a cross point switch (CPS) 13 is used as shown in FIG. 5.

A first trigger signal and a second trigger signal output from the trigger signal generator 4 shown in FIG. 1 are input to the cross point switch 13. The connection inside the cross point switch 13 is as shown in FIG. 5. Specifically, the cross point switch 13 is composed of two 1:2 connection switches that are interlocked with each other. When the two switches are connected to a side a, the first trigger signal is input to the first FET switch 11 and the second trigger signal is input to the second FET switch 12 as usual. Meanwhile, when the two switches are connected to a side b, the first trigger signal is input to the second FET switch 12 and the second trigger signal is input to the first FET switch 11, whereby polarity-inverted pulses are output.

By constantly keeping the first trigger signal in the ON state and the second trigger signal in the OFF state, switching the cross point switch 13, and storing the timing of changing the switch connection, the FET switches can be controlled. Therefore, equivalent effects can be obtained by switching the cross point switch 13 to control two trigger signals. Thus, the amount of data for control can be halved and the memory capacity can be reduced.

Incidentally, the control after completion of the driving term similar to those in Embodiments 1 and 2 can be performed by partially turning OFF trigger signals in the ON state.

### Embodiment 4

An ultrasound diagnostic device according to Embodiment 4 of the present invention has the same configuration as the ultrasound diagnostic device according to Embodiment 1, and is characterized by a frequency of a driving pulse output signal to be used and the driving timing of trigger signals, which are different from those in Embodiment 1. Specifically, a driving pulse output signal having a period TH shorter than the period TO of the characteristic vibration of the ultrasonic transducer 1 is used. In other words, the frequency of the driving pulse output signal is higher than the characteristic frequency f0 of the ultrasonic transducer 1.

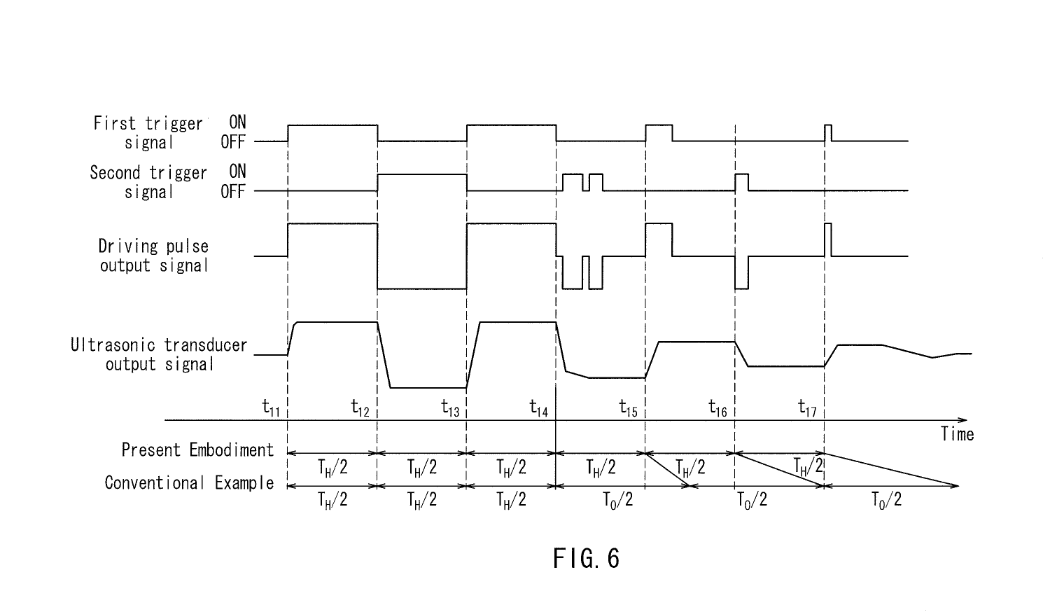

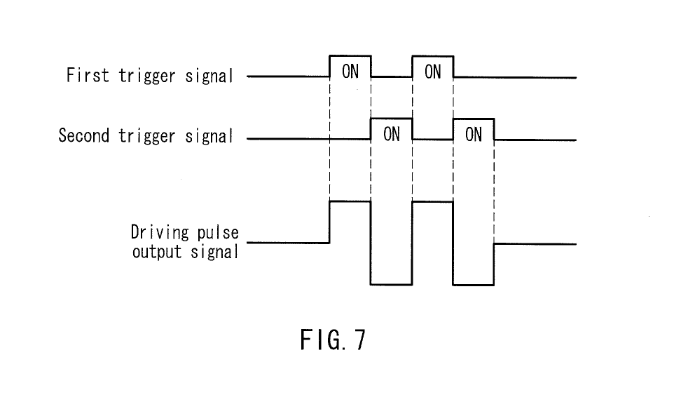

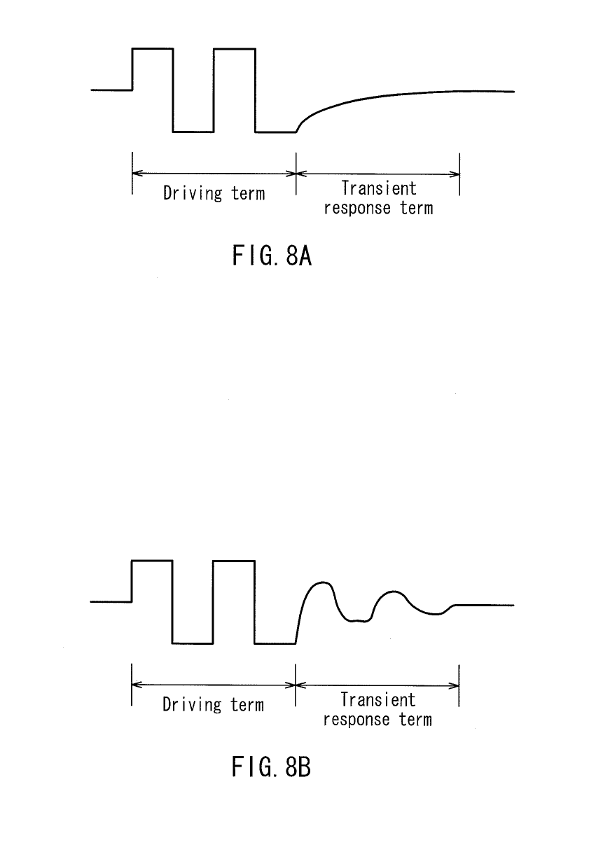

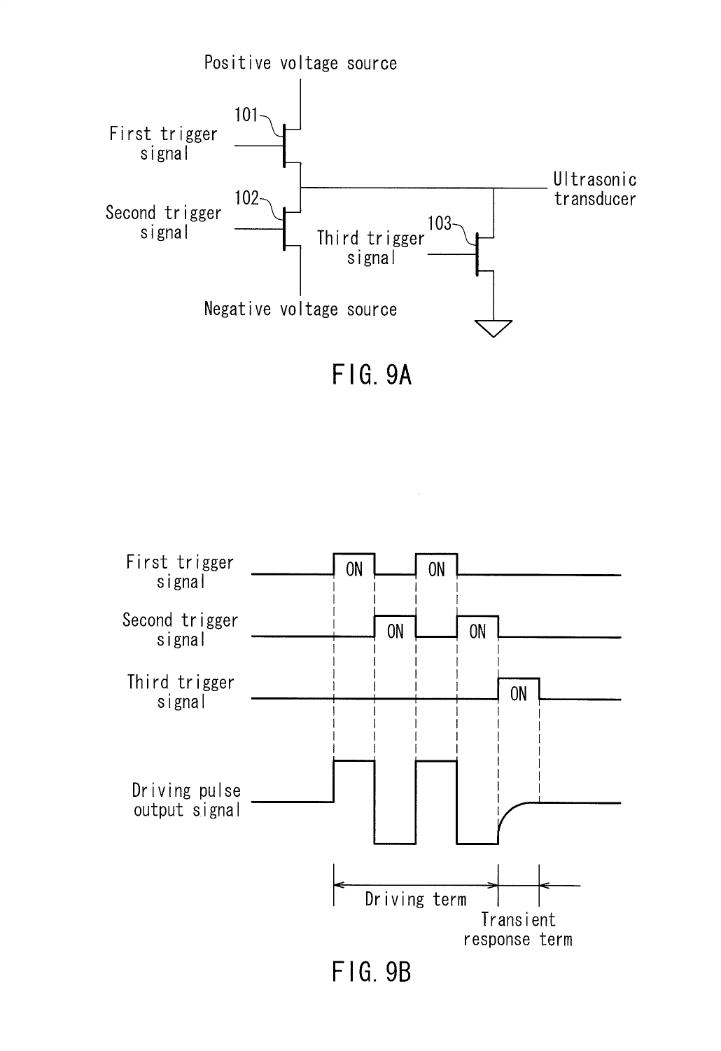

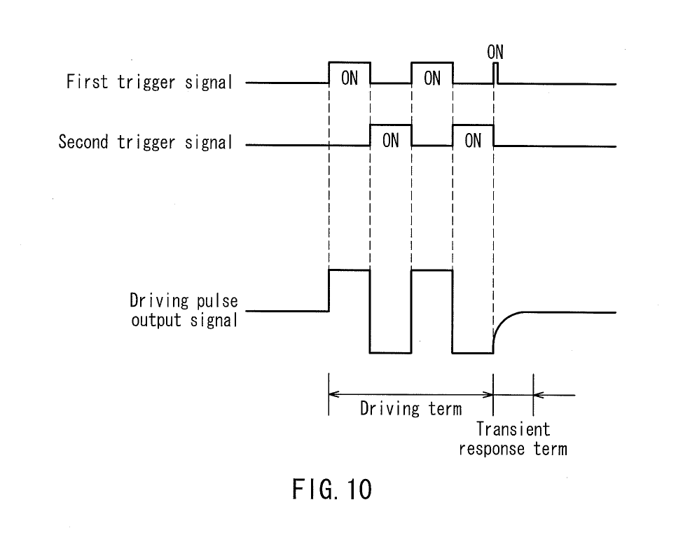

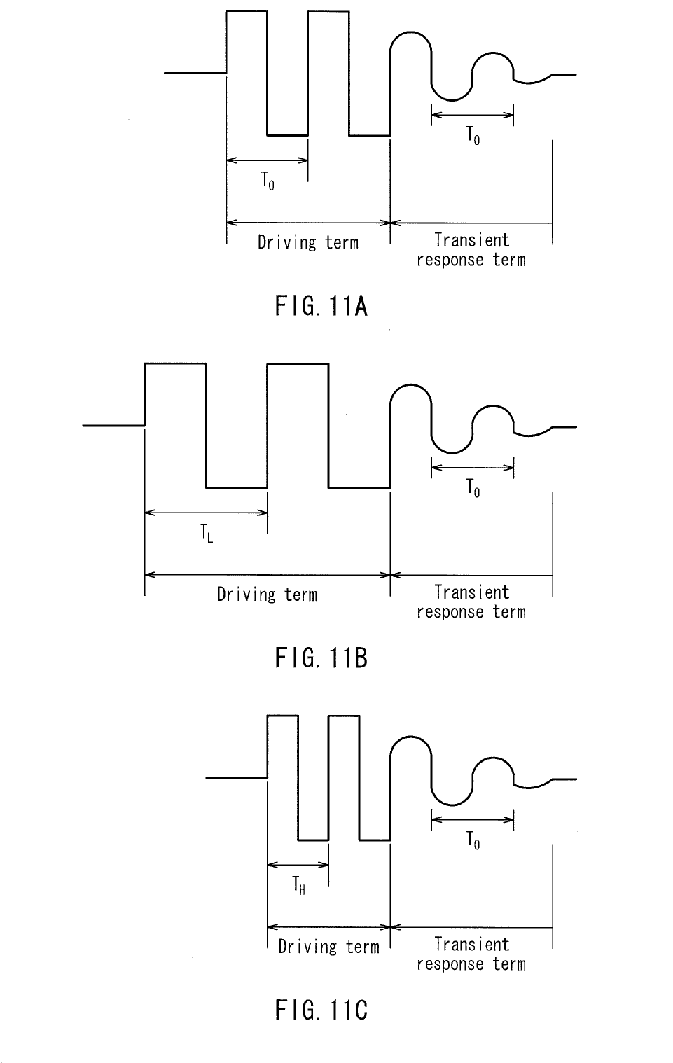

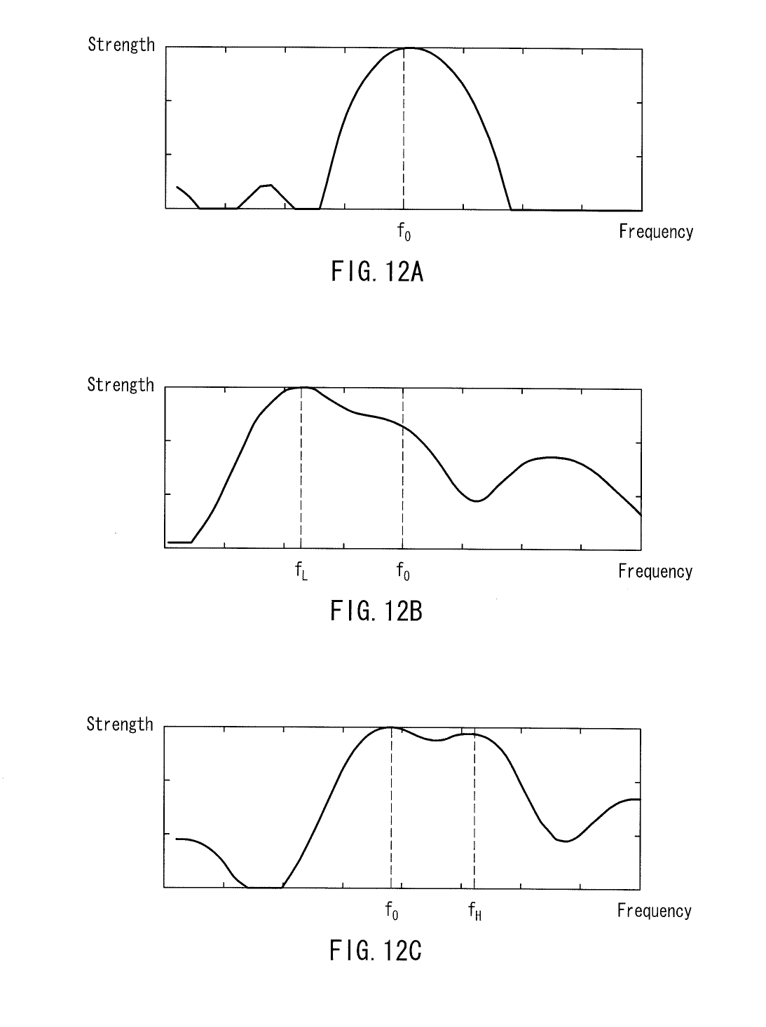

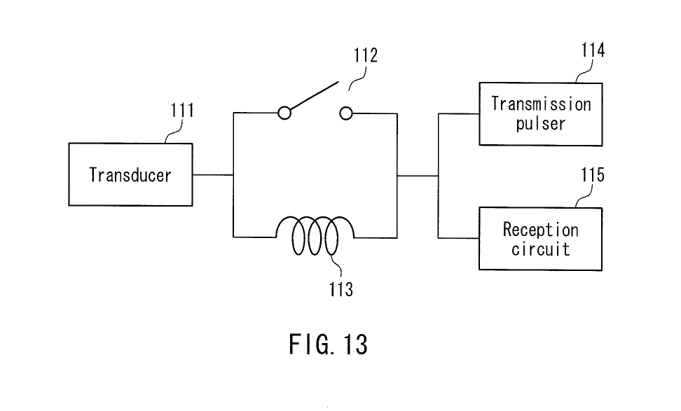

FIG. 6 is a timing chart showing a driving pulse output signal from the transmission pulser 3 of the ultrasound diagnostic device in the present embodiment, trigger signals, and an ultrasonic transducer output signal from the ultrasonic transducer 1. The driving term is from times t11 to t14, and completed at time t14. The driving pulse output signal is an output signal from the transmission pulser 3. The ultrasonic transducer output signal is an ultrasonic signal output from the ultrasonic transducer 1.

After completion of the driving term (time t14), the trigger signal generator 4 generates, as the first trigger signal and the second trigger signal, pulses shorter than TH/2. At this time, the pulse starting point, the pulse length, the number of pulses and the pause interval between pulses are adjusted so that ultrasonic transducer output signals keep the period TH while decreasing the amplitude gradually. Specifically, when a first trigger signal is ON and a second trigger signal is OFF in the term directly before completion of the driving term, the trigger signal generator 4 generates, in the next TH/2 term, a first trigger signal in the OFF state and a second trigger signal whose ON time is shorter than TH/2. Then signals are generated so that the first trigger signal and the second trigger signal are turned ON alternately per TH/2 term and the ON time is shortened gradually. Incidentally, as the second trigger signal between t14 and t15, a plurality of pulses may be used.

By generating trigger signals as described above, ultrasonic transducer output signals keep the period TH while decreasing the amplitude gradually. Because of this, frequency components lower than those in the period TH of the driving term are not generated, and the same frequency at the time of driving can be maintained. Thus, high-quality ultrasonic images with superior lateral resolution can be obtained.

### INDUSTRIAL APPLICABILITY

The present invention has an effect that irradiation of ultrasound at frequencies other than the frequency during the driving term can be suppressed also after completion of the driving term, and can be applied especially to an ultrasound diagnostic device performing harmonic imaging.

### DESCRIPTION OF REFERENCE NUMERALS

1 ultrasonic transducer
2 cable
3 transmission pulser
4 trigger signal generator
5 reception circuit
6 signal processor
7 display unit
11 first FET switch
12 second FET switch
13 cross point switch

1. An ultrasound diagnostic device comprising:
a transmission pulser that applies a driving pulse output signal to an ultrasonic transducer so as to cause the ultrasonic transducer to irradiate ultrasound; and
a trigger signal generator that generates a trigger signal that controls the transmission pulser to output the driving pulse output signal;
wherein the trigger signal generator adds, to the trigger signal in a predetermined term after completion of a driving term of the ultrasonic transducer, a plurality of trigger pulses that control an amplitude of an ultrasonic transducer output signal generated upon completion of the driving term so as to attenuate gradually.

2. The ultrasound diagnostic device according to claim 1, wherein the trigger pulses cause, in the predetermined term after completion of the driving term of the ultrasonic transducer, the ultrasonic transducer output signal generated upon completion of the driving term to keep a driving period in the driving term while attenuating the amplitude gradually.

3. The ultrasound diagnostic device according to claim 1, wherein the trigger pulses cause, in the predetermined term after completion of the driving term of the ultrasonic transducer, the ultrasonic transducer output signal generated upon completion of the driving term to attenuate the amplitude gradually by means of setting at least one of a pulse starting point, a pulse width, the number of pulses and a pulse pause term.

4. The ultrasound diagnostic device according to claim 1,
wherein the driving pulse output signal includes a positive driving pulse and a negative driving pulse, and
the transmission pulser has a first switch that generates the positive driving pulse to the ultrasonic transducer and a second switch that generates the negative driving pulse to the ultrasonic transducer.

5. The ultrasound diagnostic device according to claim 4, wherein the trigger signal includes a first trigger signal that controls the first switch and a second trigger signal that controls the second switch.

6. The ultrasound diagnostic device according to claim 5, wherein, after completion of the driving term of the ultrasonic transducer, the trigger signal brings the first switch and the second switch into an ON state alternately at 1/2 period of the driving period and shortens time of the ON state gradually per 1/2 period of the driving period.

7. The ultrasound diagnostic device according to claim 5, wherein when ON/OFF of the first switch and the second switch changes in the driving term, the trigger signal brings the first switch and the second switch into an OFF state simultaneously.

8. The ultrasound diagnostic device according to claim 5, further comprising a switching element for replacing, with each other, the first trigger signal and the second trigger signal to be input to the first switch and the second switch,
wherein pulse inversion harmonic imaging is performed by controlling the switching element.

9. The ultrasound diagnostic device according to claim 5, wherein an ON/OFF frequency of the first trigger signal and an ON/OFF frequency of the second trigger signal are lower than a center frequency of the ultrasonic transducer.

10. The ultrasound diagnostic device according to claim 5, wherein an ON/OFF frequency of the first trigger signal and an ON/OFF frequency of the second trigger signal are higher than a center frequency of the ultrasonic transducer.
